(19) United States
(12) Patent Application Publication
(10) Pub. No.: US 2010/0104647 A1
(43) Pub. Date: Apr. 29, 2010

(54) HOLLOW MICROPARTICLES

(75) Inventor: Seet Rui Simon TING, Kensington NSW (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NewSouth Innovations Pty Limited, Sydney NSW (AU)

(21) Appl. No.: 12/526,247

(22) PCT Filed: Feb. 8, 2008
(86) PCT No.: PCT/AU08/00158

§ 371 Date: Nov. 9, 2009

(30) Foreign Application Priority Data
Feb. 9, 2007 (AU) 2007900647

(51) Int. Cl. A61K 9/14; C08J 9/00; C08F 2/46; A61K 47/32; A61P 35/00

(52) U.S. Cl. 424/489; 523/218; 522/167; 522/182; 514/772.5; 514/772.4

(57) ABSTRACT

The invention provides a process for making hollow microparticles. The process comprises providing a dispersion having a continuous aqueous phase and a discontinuous organic phase and polymerising a monomer in the dispersion to form hollow polymeric microparticles. The continuous aqueous phase of the dispersion comprises a stabiliser and the discontinuous organic phase of the dispersion comprises the monomer and an organic liquid. The monomer has two or more polymerisable groups per molecule. Prior to the step of polymerising the monomer, the discontinuous organic phase does not contain a polymer.

### TECHNICAL FIELD

The present invention relates to a process for making hollow microparticles.

### BACKGROUND OF THE INVENTION

Hollow microparticles have a range of potential applications. The empty core allows the encapsulation of a range of material in high concentration. Possible applications therefore include drug delivery and catalysis. Microparticles are, for example, of great interest as a drug delivery system, in particular for drug administration through the hepatic artery for treatment of liver tumours.

Currently hollow spheres are employed in industry as insulating materials, light-weight materials and materials with reduced electrical and heat conductivity. Most hollow spheres, especially those in industry, are processed from inorganic materials such as ceramics, TiO₂, Y₂O₃, SiO₂ and glass. (J. Breitling, J. Bloemer, R. Kuemmel, Chem. Eng. Technol. 2004, 27, 829.) Techniques to prepare these particles are manifold and range from high temperature smelting to spray and dripping technologies, emulsion and suspension processes. Many techniques are solely applied for inorganic materials.

Hollow polymer microspheres can be prepared by a range of techniques, but the range of options is limited compared to inorganic particles. Most commonly, a template is coated by a polymer layer followed by removal of the template. Templates such as polystyrene or SiO₂ are either removed by solvent treatment or etching with HF. A range of other more unusual templating techniques have been used, such as micellar systems or vesicles.

It seems apparent that the synthesis of hollow spheres without the sacrifice of a templating core is desirable. In situ techniques not only avoid the necessary synthesis of a suitable core, they also eliminate the tedious task of removing the core, which can cause problems.

A range of non-templating techniques have been used. Poly(o-toluidine) microspheres were prepared by suspending the monomer in water followed by oxidative polymerization. The resulting hollow spheres had usually sizes below 10 micron. In addition every sphere was observed to have one hole.

Vesicles (or niosomes, polymerosomes) are naturally hollow spheres composed of bilayers. Crosslinking of these layers allow the stabilisation of these aggregates. The size of these structures is, however, limited to 10 microns.

Emulsion processes employ a water/oil system. The polymer precipitates along the interface between two phases forming hollow spheres. This system has been utilized in the preparation of hollow amino resins by suspending a precondensate followed by a heating step. Another technique uses the arrangement of polymerizable polymers along the interface between oil and water phase. Subsequent interfacial polymerization of these macromers results in hollow spheres. However, the limited surface thickness (typically nanoscale) does not provide sufficient stability. Thus, the spheres easily collapse.

Hollow microspheres have been manufactured by a solvent evaporation process. The polymer was dissolved in a suitable non-polar solvent. After suspending the oil droplets in water, the solvent was allowed to evaporate.

Emulsion techniques seem to provide a versatile way to prepare hollow microcapsules. However, the techniques described above have limitations regarding the stability of the resulting particles. The particles either collapse easily due to insufficient thickness of the wall or surface erosion occurs when these spheres are employed under theologically demanding conditions.

A technique has been proposed for using suspension polymerization in combination with an oil phase, which presents a non-solvent for the polymer. The synthesis of hollow spheres, using mainly divinylbenzene (DVB) as monomer, and a toluene/water system containing preformed polymer was described and investigated in detail. In the initial stages of the polymerization the monomer is soluble in the polymer-containing toluene droplet. With increasing conversion and the formation of PDVB, phase separation occurs and the polymer precipitates along the interface. According to the empirical relationship for suspension polymerization between particle size and polymerization parameter this technique should be suitable to prepare hollow capsules within a large size range. However, the microsphere size reported was usually around 10 microns.

The use of divinyl benzene is limited to capsules which are expected to have a high durability. Degradation and surface erosion is limited. Nevertheless, many applications require the synthesis of stimuli-responsive or degradable systems. There is a need for a process for making hollow microspheres which are capable of degrading over time.

### OBJECT OF THE INVENTION

It is the object of the present invention to substantially overcome or at least ameliorate one or more of the above disadvantages.

### SUMMARY OF THE INVENTION

In a first aspect of the invention there is provided a process for making hollow microparticles comprising:
a) providing a dispersion having a continuous aqueous phase and a discontinuous organic phase, wherein the continuous aqueous phase comprises a stabiliser and the discontinuous organic phase comprises a monomer having two or more polymerisable groups per molecule and an organic liquid; and
b) polymerising the monomer in the dispersion to form hollow polymeric microparticles.

The dispersion may be an emulsion. It may be a suspension. The dispersion may be an oil in water emulsion. The dispersion may be an oil in water suspension.

In an embodiment of this aspect, prior to step b) the discontinuous organic phase does not contain a polymer.

The monomer may be selected so that the polymer of the polymeric microparticles is capable of reacting with a chemical to which the microparticles are exposed in order to release a substance encapsulated in the hollow microparticles. The monomer may comprise a cleavable, e.g. hydrolysable, linkage between two of the polymerisable groups, such that the polymer is capable of cleaving, e.g. hydrolysing, in order to release the substance. Thus the monomer may be selected so that the polymer of the polymeric microparticles is capable of reacting with a cleaving agent, e.g. a hydrolysing agent (e.g. an acid or a base or a hydrolytic enzyme or a thiol) in order to release a substance encapsulated in the hollow microparticles. The cleavable, e.g. hydrolysable, linkage may comprise an ester group, an anhydride group, an orthoester group, an acetal group, a disulfide group or may comprise more than one of these, or some other cleavable, e.g. hydrolysable, group. Thus the cleavable linkage may comprise for example a disulfide. If the cleavable linkage is a hydrolysable linkage then it may comprise for example an ester group, an anhydride group, an orthoester group, an acetal group. The monomer may comprise a cleavable, e.g. hydrolysable, group (such as those above), such that cleavage, e.g. hydrolysis, of the cleavable, e.g. hydrolysable, group in the polymer alters the polarity of the polymer without causing backbone chain breaking of the polymer. In this case, the cleavable, e.g. hydrolysable, group may be in a side chain of the polymer.

The stabiliser may be polymeric. It may be a thickener. It may be for example polyvinylpyrrolidone (PVP), polyvinyl alcohol (PVA) or a mixture of these.

The organic liquid may be a non-solvent for the polymer formed in step b). The organic liquid may be a hydrophobic organic liquid. The organic liquid may be incapable of swelling the polymer. The solubility of the polymer in the organic liquid at the temperature of step b) may be less than about 1% w/v, or between about 0 and 1% w/v. The organic liquid may be a solvent for the monomer. The organic liquid may be both a non-solvent for the polymer and a solvent for the monomer. The monomer may be soluble in the organic liquid and the polymer may be insoluble in the organic liquid. The polymer may be non-swellable by the organic liquid. In step a) the monomer may be dissolved in the organic liquid. The organic liquid may be a non-polymerisable liquid. It may be incapable of copolymerising with the monomer.

The discontinuous organic phase may additionally comprise a second monomer, or further monomers, which are capable of copolymerising in step b) with the monomer having two or more polymerisable groups per molecule, thereby forming hollow polymeric microparticles wherein the polymer of the microparticles is a copolymer. The second or further monomers may be non-crosslinking second or further monomers. They may have a single polymerisable group per molecule. The second monomer, or at least one of the further monomers, may be such that groups in the copolymer are capable of reacting, e.g. hydrolysing, so as to render the surface of the microparticles hydrophilic. In the event that a second monomer, or further monomers, are present in the discontinuous phase, said second monomer or further monomers may be soluble in the organic liquid. They may be such that the copolymer formed by copolymerising the monomer having two or more polymerisable groups per molecule with said second monomer or further monomers is insoluble in the organic liquid.

Step b) may comprise polymerising the monomer in the dispersed organic phase to form hollow polymeric microparticles. The monomer having two or more polymerisable groups per molecule may be polymerisable by thermally initiated polymerisation. It may be polymerisable by radiation initiated polymerisation (UV, gamma ray, e-beam etc.). Thus the discontinuous organic phase may comprise a thermal initiator and step b) may then comprise heating the dispersion so as to polymerise the monomer. The discontinuous organic phase may comprise a photoinitiator and step b) may then comprise irradiating the dispersion with radiation (e.g. UV, gamma ray, e-beam etc.) having a wavelength and intensity sufficient to polymerise the monomer. The thermal initiator and/or the photoinitiator, if present, may be soluble in the discontinuous organic phase, e.g. in the monomer, the organic liquid, or both. It may be insoluble in the continuous aqueous phase.

The microparticles may comprise an interior region surrounded by a polymeric shell. The polymeric shell may comprise a polymer which is insoluble in the organic liquid. The polymer may be derived by polymerisation, or by copolymerisation, of the monomer.

The process may additionally comprise:
b') at least partially reacting groups in the polymeric shell derived from a second or further monomer so as to render the surface of the microparticles hydrophilic.

The reacting may comprise hydrolysing. Thus for example, the second or further monomer may comprise a hydrolysable group such as an ester group, and hydrolysis thereof may provide a hydrophilic group on the surface of the microparticles.

In some embodiments the monomer having two or more polymerisable groups per molecule comprises a degradable non-hydrolysable linking group and the second monomer comprises a hydrolysable group, whereby a shell of the hollow microparticles comprises a copolymer comprising degradable non-hydrolysable crosslinks and also comprising hydrolysable groups. In this case the process may comprise at least partially hydrolysing the hydrolysable groups of the copolymer so as to render the surface of the microparticles hydrophilic.

The process may additionally comprise:
c) loading a substance into the interior region of the hollow microparticles to form loaded microparticles.

The substance may be a drug. It may be usable in the treatment of cancer. It may be capable of generating a β-emitter when subjected to neutron bombardment.

In another embodiment the process comprises:
a) providing a dispersion having a continuous aqueous phase and a discontinuous organic phase, wherein the continuous aqueous phase comprises a polymeric stabiliser and the discontinuous organic phase comprises a monomer having two or more polymerisable groups per molecule, said monomer being dissolved in an organic liquid; and
b) polymerising the monomer in the discontinuous organic phase to form hollow polymeric microparticles, said microparticles comprising an interior region surrounded by a shell comprising a polymer which is insoluble in the organic liquid,
   wherein prior to step b) the discontinuous organic phase does not contain a polymer.

In another embodiment the process comprises:
a) providing a dispersion having a continuous aqueous phase and a discontinuous organic phase, wherein the continuous aqueous phase comprises a polymeric stabiliser and the discontinuous organic phase comprises a monomer having two or more polymerisable groups per molecule, said monomer being dissolved in an organic liquid;
b) polymerising the monomer in the discontinuous organic phase to form hollow polymeric microparticles, said microparticles comprising an interior region surrounded by a shell comprising a polymer which is insoluble in the organic liquid; and
c) loading a substance into the interior region of the hollow microparticles to form loaded microparticles
   wherein prior to step b) the discontinuous organic phase does not contain a polymer.
   The dispersion may be an oil in water dispersion.

In another embodiment the process comprises:
a) providing an oil in water dispersion having a continuous aqueous phase and a discontinuous hydrophobic organic phase, wherein the continuous aqueous phase comprises a polymeric stabiliser and the discontinuous hydrophobic organic phase comprises a monomer having two or more polymerisable groups per molecule, said monomer being dissolved in a hydrophobic organic liquid;
b) polymerising the monomer in the discontinuous hydrophobic organic phase to form hollow polymeric microspherical particles, said microspherical particles comprising an interior region surrounded by a shell comprising a polymer which is insoluble in the hydrophobic organic liquid; and
c) loading a substance into the interior region of the hollow microspherical particles to form loaded microspherical particles,
   wherein prior to step b) the discontinuous hydrophobic organic phase does not contain a polymer.

In a further embodiment the process comprises:
a) providing a dispersion having a continuous aqueous phase and a discontinuous organic phase, wherein the continuous aqueous phase comprises a polymeric stabiliser and the discontinuous organic phase comprises a first monomer having two or more polymerisable groups per molecule and a second monomer copolymerisable with the first monomer, said first and second monomers being dissolved in an organic liquid; and
b) copolymerising the first and second monomers in the discontinuous organic phase to form hollow polymeric microparticles, said microparticles comprising an interior region surrounded by a shell comprising a polymer which is insoluble in the organic liquid,
   wherein prior to step b) the discontinuous organic phase does not contain a polymer.

In a further embodiment the process comprises:
a) providing a dispersion having a continuous aqueous phase and a discontinuous organic phase, wherein the continuous aqueous phase comprises a polymeric stabiliser and the discontinuous organic phase comprises a first monomer having two or more polymerisable groups per molecule and a second monomer copolymerisable with the first monomer, said first and second monomers being dissolved in an organic liquid;
b) copolymerising the first and second monomers in the discontinuous organic phase to form hollow polymeric microparticles, said microparticles comprising an interior region surrounded by a shell comprising a polymer which is insoluble in the organic liquid; and
c) at least partially reacting groups in the polymer of the shell derived from the second monomer so as to render the surface of the microparticles hydrophilic,
   wherein prior to step b) the discontinuous organic phase does not contain a polymer.

In a further embodiment the process comprises:
a) providing a dispersion having a continuous aqueous phase and a discontinuous organic phase, wherein the continuous aqueous phase comprises a polymeric stabiliser and the discontinuous organic phase comprises a first monomer having two or more polymerisable groups per molecule and a second monomer copolymerisable with the first monomer, said first and second monomers being dissolved in an organic liquid;
b) copolymerising the first and second monomers in the discontinuous organic phase to form hollow polymeric microparticles, said microparticles comprising an interior region surrounded by a shell comprising a polymer which is insoluble in the organic liquid;
c) at least partially reacting groups in the polymer of the shell derived from the second monomer so as to render the surface of the microparticles hydrophilic; and
d) loading a substance into the interior region of the hollow microspherical particles to form loaded microspherical particles,
   wherein prior to step b) the discontinuous organic phase does not contain a polymer.

The invention also provides hollow microparticles made by the process of the first aspect, including any of the embodiments thereof. The microparticles may be dispersible in an aqueous liquid, e.g. in water.

In a second aspect of the invention there is provided a method of treating a condition in a patient comprising administering to said patient a therapeutically effective quantity of microparticles according to the invention, wherein a substance indicated for treatment of said condition is located in the interior region of the microparticles. The microparticles may be made by the process of the invention, including any of the embodiments thereof.

The method may be a method of treating cancer in a patient, comprising:
exposing a therapeutic quantity of hollow polymeric microparticles according to the invention to neutrons from a neutron source, wherein a substance in the interior region of the hollow microparticles is such that the exposure generates a β-emitter; and
administering the therapeutic quantity of the hollow microparticles to the patient.

In a third aspect of the invention there is provided the use of hollow polymeric microparticles according to the invention for the manufacture of a medicament for the treatment of cancer. The cancer may be for example liver cancer or it may be some other cancer. There is also provided the use of hollow polymeric microparticles according to the invention for the treatment of cancer, wherein a substance indicated for treatment of cancer is located in the interior region of the microparticles. There is also provided hollow polymeric microparticles according to the invention when used for treating cancer, wherein a substance indicated for treatment of cancer is located in the interior region of the microparticles.

The invention also provides hollow polymeric microparticles comprising a polymeric shell surrounding a non-polymeric interior region (core). The core may be hollow. It may contain gas, liquid or a non-polymeric solid, or may contain more than one of these. The core may contain an active material, e.g. a biologically active material. The polymeric shell may be a crosslinked polymeric shell. It may comprise a copolymer. It may comprise a homopolymer. The crosslinked polymeric shell may comprise cleavable, e.g. hydrolysable, crosslinks. The hollow microparticles may be cleavable, e.g. hydrolysable, so as to release a substance located in the core. The microparticles may be dispersible in an aqueous liquid, e.g. in water. The microparticles may be dispersible in a polar liquid, e.g. an alcohol such as ethanol or methanol. The microparticles may be dispersible in a nonpolar liquid. The microparticles may have hydrophilic surfaces. They may have hydrophobic surfaces. They may have surfaces which vary in hydrophilicity as a function of pH and/or temperature and/or other stimulus from the environment.

In a fourth aspect of the invention there is provided a method for releasing a substance from a core of a polymeric microparticle according to the present invention, said core containing the substance, said method comprising exposing said polymeric microparticle to a reagent which causes cleavage of crosslinks of a polymeric shell of the microparticle so as to cause said microparticle to release said substance.

The crosslinks which are cleaved by the reagent may be bonds derived from the monomer having two or more polymerisable groups per molecule. They may be cleavable crosslinks. They may be hydrolysable crosslinks. The reagent may be a hydrolytic reagent (e.g. a base or an acid) in the case where the crosslinks are hydrolysable crosslinks (e.g. esters, amides or anhydrides) or may be a reductive reagent in the case that the crosslinks are reducible crosslinks (e.g. a disulfides).

Thus in some embodiments said crosslinks are hydrolysable crosslinks and said reagent is a hydrolytic reagent. In other embodiments said crosslinks are disulfide crosslinks and said reagent comprises a thiol.

### DETAILED DESCRIPTION OF THE INVENTION

The process of the present invention is provided for making hollow polymeric microparticles, which may be used for example in therapeutic applications including delivery of drugs and in treatment of cancer. The process comprises providing a dispersion in which a discontinuous organic phase is dispersed in a continuous aqueous phase. The discontinuous organic phase comprises a monomer, which is polymerised in the droplets of the discontinuous organic phase to provide hollow polymeric microparticles. The hollow microparticles comprise a polymeric shell surrounding a hollow core. In particular they comprise a crosslinked polymeric shell surrounding a hollow core. Commonly the continuous aqueous phase and the discontinuous organic phase are liquid phases. The dispersion may be an oil in water dispersion.

The dispersion may comprise a surfactant. There may be no surfactant in the dispersion. It may be a suspension. It may be an emulsion. The dispersion, prior to step b) may be a stable dispersion. It may be an unstable dispersion. In the absence of external agitation, it may show no separation for up to about 30 seconds, or up to about 1, 2, 3, 4, 5, 10, 15, 20, 25, 30, 40, 50 or 60 minutes, or up to 1, 2, 6, 12 or 24 hours, or up to 1, 2, 3, 4, 5, 10, 15, 20, 25 or 30 days, or for more than 30 days. In the present process the dispersion may be agitated e.g. stirred, shaken, sonicated, swirled etc. This may serve to maintain the dispersion. The dispersion may have a narrow distribution of droplet sizes. It may have a broad distribution of droplet sizes. It may be substantially monodispersed. The dispersion may have a polydispersity of droplet sizes (defined as weight average droplet size divided by number average droplet size) of between about 1 and about 10, or between about 1 and 5, 1 and 4, 1 and 3, 1 and 2, 1 and 1.5, 1 and 1.2, 2 and 10, 5 and 10 or 2 and 5, e.g. about 1, 1.1, 1.2, 1.3, 1.4, 1.5, 1.6, 1.7, 1.8, 1.9, 2, 2.5, 3, 3.4, 4, 4.5, 5, 6, 7, 8, 9 or 10, or more than about 10. The droplets may have a mean diameter between about 0.1 microns and about 2 mm or between about 0.1 microns and 1 mm, 0.1 and 500 microns, 0.1 and 200 microns, 0.1 and 100 microns, 0.1 and 50 microns, 0.1 and 10 microns, 0.1 and 5 microns, 0.1 and 1 micron, 1 micron and 2 mm, 10 microns and 2 mm, 50 microns and 2 mm, 100 microns and 2 mm, 200 microns and 2 mm, 500 microns and 2 mm, 1 and 2 mm, 1 and 500 microns, 1 and 200 microns, 1 and 100 microns, 1 and 50 microns, 1 and 20 microns, 1 and 10 microns, 10 and 500 microns, 50 and 500 microns, 100 and 500 microns, 200 and 500 microns, 10 and 200 microns, 10 and 100 microns, 10 and 75 microns or 10 and 50 microns. For example, the droplets may have a mean diameter of about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800 or 900 microns, or about 1, 1.1, 1.2, 1.3, 1.4, 1.5, 1.6, 1.7, 1.8, 1.9 or 2 mm.

The continuous aqueous phase comprises water and a stabiliser. It may also contain additional components, e.g. salts, which may be in solution in the aqueous phase. The stabiliser may be a dispersion stabiliser, e.g. an emulsion stabiliser or a suspension stabiliser. It may be a water soluble stabiliser. It may be a water insoluble stabiliser. It may be a hydrophilic stabiliser. The stabiliser may be present in sufficient concentration to provide the desired stability of the dispersion (as described above). It may be present in sufficient concentration to provide a desired aqueous phase viscosity, i.e. it may be a viscosity modifier. The stabilizer may act as a barrier between droplets and thereby prevent or inhibit coagulation of droplets in the dispersion,

The desired aqueous phase viscosity may be between about 0.4 and about 1000cS at the temperature at which polymerisation is conducted. or between about 1 and 500, 1 and 200, 1 and 100, 1 and 50, 1 and 20, 1 and 10, 1 and 5, 5 and 1000, 50 and 1000, 100 and 1000, 500 and 1000, 5 and 500, 5 and 100, 5 and 50, 10 and 100 or 50 and 100cS, e.g. about 1, 1.5, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900 or 1000cS, or more than 1000cS, or it may be less than 1cS, e.g. about 0.9, 0.8, 0.7, 0.6, 0.5, 0.4 or 0.3cS, or between about 0.3 and 1, 0.3 and 0.5, 0.5 and 1 or 0.4 and 0.8cS. The stabiliser may be a polymer. It may be for example PVP or PVA or a polycarboxylic acid salt or a polyamine or some other polyelectrolyte. The PVA may have a degree of hydrolysis sufficient to be water soluble. The degree of hydrolysis may be between about 80 and 100%, or between about 80 and 90, 80 and 85, 85 and 100, 90 and 100, 95 and 100 or 85 and 95%, e.g. about 80, 81, 82, 83, 84, 85, 86, 87, 88, 89, 90, 91, 92, 93, 94, 95, 96, 97, 98, 99 or 100%. The stabiliser may be an inorganic stabiliser. It may for example be clay, silica (e.g. silica gel) or a salt. The stabiliser may be present in the aqueous phase at between about 0.05 and about 1% by weight, or between about 0.1 and 1, 0.2 and 1, 0.5 and 1, 0.05 and 0.5, 0.05 and 0.2, 0.05 and 0.1, 0.1 and 0.5 or 0.3 and 0.7, e.g. about 0.05, 0.1, 0.15, 0.2, 0.25, 0.3, 0.34, 0.4, 0.45, 0.5, 0.55, 0.6, 0.65, 0.7, 0.75, 0.8, 0.85, 0.9, 0.95 or 1%, or some other concentration. The concentration of the stabiliser may depend on the nature of the stabiliser (i.e. chemical nature, molecular weight etc.).

The continuous aqueous phase may represent between about 80 and about 99% of the dispersion by weight or by volume, or between about 80 and 95, 80 and 90, 85 and 99, 90 and 99, 95 and 99, 85 and 95, 92 and 97, 85 and 90 or 90 and 95%, e.g. about 80, 81, 82, 83, 84, 85, 86, 87, 88, 89, 90, 91, 92, 93, 94, 95, 96, 97, 98 or 99%.

The discontinuous organic phase may represent between about 1 and about 20% of the dispersion by weight or volume, or between about 1 and 15, 1 and 10, 1 and 5, 1 and 2, 2 and 20, 5 and 20, 10 and 20, 2 and 10 or 3 and 7%, e.g. about 1, 2, 3, 4, 5, 6, 7, 8, 910, 11, 12, 13, 14, 1,5 16, 17, 18, 19 or 20%.

The discontinuous organic phase comprises a monomer having two or more polymerisable groups per molecule. It may have for example 2, 3, 4, 5 or more than 5 polymerisable groups per molecule. The groups may be olefinic groups e.g. acrylic or methacrylic groups, optionally substituted. The concentration of this monomer in the discontinuous phase may be between about 5 and about 75% w/w or v/v, or between about 5 and 60, 5 and 50, 5 and 40, 5 and 30, 5 and 25, 5 and 10, 10 and 75, 25 and 75, 50 and 75, 20 and 70, 30 and 60, 40 and 60 or 45 and 55%, e.g. about 5, 10, 15, 20, 25, 30, 35, 40, 45, 50, 55, 60, 65, 70 or 75%.

The monomer may be selected so that the polymer is capable of reacting with a chemical to which the microparticles are exposed in order to release a substance encapsulated in the hollow microparticles. Thus release of the encapsulated substance may be controlled by controlling the exposure of the microparticles to the chemical. The chemical may comprise an acid, an enzyme (e.g. a hydrolytic enzyme), a base or some other chemical. The monomer may comprise a hydrolysable linkage between two of the polymerisable groups. In this case the polymer is capable of hydrolysing in order to release the substance.

The hollow microparticles may be responsive to pH changes in the environment by either of the following mechanisms:
a) The capsules disintegrate fully at varying rates depending on the structure of the crosslinker. The class of crosslinkers proposed includes disulfides, esters, anhydrides, orthoester and acetals.

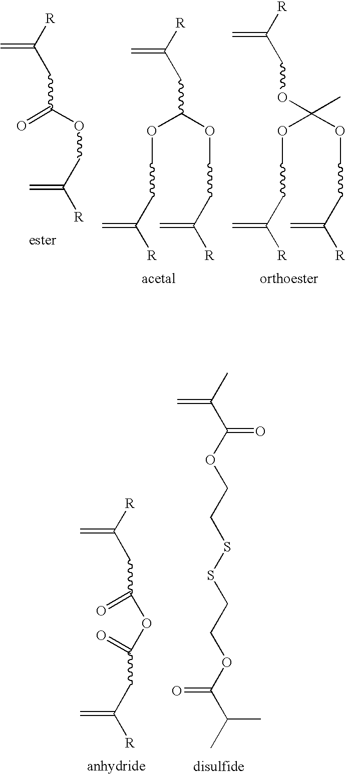

b) The capsule changes its polarity (irreversibly) with changes in pH value by cleavage of crosslinker side chains or side groups, or of side chains or side groups derived from second or further monomers (e.g. non-crosslinking second or further monomers). The hollow capsule remains intact, but polarity changes allow now the penetration of water and polar compounds.

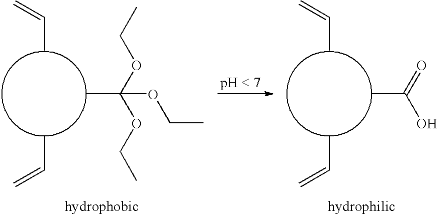

The polymerisable groups may be connected by a linker. The linker may be hydrolysable. It may for example comprise an ester group, an anhydride group, an orthoester group, an acetal group, a thioester group, a carbonate group, a thiocarbonate group, a dithiocarbonate group, a trithiocarbonate group, a urethane group, an amide group or some other hydrolysable group. The group may be slowly hydrolysable. It may be rapidly hydrolysable. The linker may be hydrophilic. It may for example comprise an ethylenedioxy group or an oligo- or poly-ethylene oxide group, which may be coupled to the polymerisable groups through ester linkages or other suitable hydrolysable linkages, Examples include ethylene glycol dimethacrylate or diacrylate, diethylene glycol dimethacrylate or diacrylate, polyethylene glycol dimethacrylate or diacrylate etc. When a monomer comprising two or more polymerisable groups connected by a hydrolysable linker is polymerised, the resulting polymer will have hydrolysable crosslinks comprising the hydrolysable linker. Hydrolysis of the crosslinks may then lead to degradation of the polymer.

In an alternative, the monomer may comprise a hydrolysable group that is not present in a linker group connecting two or more polymerisable groups. In this case, when the monomer is polymerised, hydrolysis of the hydrolysable group does not cause cleavage of the polymer backbone. However such hydrolysis may lead to a change in the polarity of the polymer due to the hydrolysis. Thus for example if the monomer comprises a trialkyl orthoester group, hydrolysis of the resulting polymer converts the orthoester to a carboxylic acid, thereby increasing the polarity of the polymer.

The monomer may comprise polymerisable groups connected by a hydrolysable linker and a hydrolysable group that is not present in a linker group connecting two or as more polymerisable groups.

The discontinuous organic phase may additionally comprise one or more further monomers (e.g. 2, 3, 4, 5 or more than 5). If present, these should be capable of copolymerising with the monomer having two or more polymerisable groups per molecule. They may be unsaturated monomers, and may be acrylates, methacrylates, acrylamides, methacrylamides, vinyl ethers, styrenic monomers or some other suitable type of monomer. They may have a single polymerisable (or copolymerisable) group per molecule. The concentration of all of the further monomers combined in the discontinuous organic phase may be between about 0 and about 95%, or between about 0 and 90, 0 and 70, 0 and 50, 0 and 40, 0 and 30, 0 and 20, 0 and 10, 0 and 5, 1 and 50, 1 and 40, 1 and 30, 1 and 20, 1 and 10, 1 and 5, 1 and 2, 5 and 50, 10 and 50, 25 and 50, 5 and 25, 5 and 10, 20 and 25, 10 and 95, 25 and 95, 50 and 95, 70 and 95, 20 and 90, 50 and 90 or 30 and 70, e.g. about 0, 1, 2, 3, 4, 5, 10, 15, 20, 25, 30, 35, 40, 45, 60, 70, 80, 90 or 95%. It will be understood that if further monomers are present in the dispersion prior to step b), then the polymer formed in step b) will be a copolymer comprising monomer units derived from the monomer having two or more polymerisable groups per molecule and monomer units derived from the one or more further monomers.

The discontinuous organic phase also comprises an organic liquid. The discontinuous organic phase may comprise a hydrophobic organic liquid. The organic liquid may be a non-solvent for the polymer. It may be a non-polymeric organic liquid. It may be a non-polymerisable organic liquid. It may be an organic liquid that is incapable of copolymerising with the monomer having two or more polymerisable groups per molecule or with the second and further monomers if present. The solubility of the polymer in the organic liquid at the temperature of step b) may be sufficiently low that, when the polymer forms by polymerisation of the monomer (optionally by copolymerisation with the further monomers), the polymer forms as a shell at and/or near the outside surface of the droplets of the discontinuous organic phase. The solubility of the polymer in the organic liquid may be less than about 1% w/w or w/v. The polymer, or copolymer, may be sufficiently polar that it forms at and/or near the interface between the continuous aqueous phase and the discontinuous organic phase, thereby forming a hollow microparticle. The monomer (or the mixture of monomers and further monomers if the latter are present) may be sufficiently polar that the polymer forms at and/or near the interface between the continuous aqueous phase and the discontinuous organic phase. The polymer may be more polar than the solvent. The monomer (or the mixture of monomers and further monomers if the latter are present) may be such (i.e. may be sufficiently polar) that the polymer is more polar than the solvent. The linker in the monomer may be sufficiently polar that the monomer (or the mixture of monomers and further monomers if the latter are present) is such that the polymer is more polar than the solvent. The organic liquid may be a solvent for the monomer, and additionally for the further monomers if present. The solubility of the monomer and, if present, further monomers, in the organic liquid may be sufficient that the discontinuous organic phase prior to step b) is a solution, optionally a homogeneous solution (e.g. having no undissolved components). The organic liquid may immiscible with water. It may be only slightly miscible with water. It may be hydrophobic. It may be for example an ester, an ether, a ketone, an aromatic hydrocarbon or some other type of organic liquid. It may be cyclic. It may be acyclic. Suitable organic liquids include butyl acetate and ethyl acetate. The organic liquid may be, or may comprise, a polymerisable compound that polymerizes slower than the monomer. In this option, the polymerisable compound may such that it polymerises to form a soluble polymer. In an example, vinylneodecanoate (VND) is used as the organic liquid (either alone or in combination with some other organic liquid) and ethylene glycol dimethacrylate (EGDMA) is used as the monomer. VND is a polymerisable substance, which slowly polymerizes and thereby forms a solid core that can be washed out to form a hollow microparticle. In this case, in early stages of the polymerisation, EGDMA is polymerizes to form a shell and in later stages VND polymerises to form a polymeric core within the shell. In intermediate stages of the polymerisation is a transition in which VND and EGDMA copolymerize. The result is a shell that consists mainly of polymerised EGDMA, but also a small fraction of copolymerised VND.

Following polymerisation of the monomer (step (b)), the resulting polymer is in the form of hollow microparticles. Following polymerisation of the monomer (step (b)), the resulting polymer is in the form of hollow spherical microparticles. Following polymerisation of the monomer (step (b)), the resulting polymer is in the form of hollow pseudospherical microparticles. The polymer may be insoluble in the organic liquid. It may be of low solubility in the organic liquid. It may be of sufficiently low solubility in the organic liquid that the polymer as it forms in step (b) forms in the shape of a shell towards the outside of the droplets of the dispersion so as to form hollow microparticles. The solubility of the polymer in the organic liquid may be less than about 1% (w/w or w/v) or less than about 0.5, 0.2 or 0.1%, or between about 0 and about 1%, or between about 0 and 0.5, 0 and 0.2, 0 and 0.1, 0 and 0.05, 0 and 0.01, 0.1 and 1, 0.5 and 1 or 0.1 and 0.5, e.g. about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9 or 1%. The organic liquid may be a non-swelling liquid for the polymer. It may be sufficiently non-swelling for the polymer that the polymer as it forms in step (b) forms in the shape of a shell towards the outside of the droplets of the dispersion so as to form hollow microparticles. The organic liquid may swell the polymer less than about 1% (w/w or w/v) or less than about 0.5, 0.2 or 0.1%, or between about 0 and about 1%, or between about 0 and 0.5, 0 and 0.2, 0 and 0.1, 0 and 0.05, 0 and 0.01, 0.1 and 1, 0.5 and 1 or 0.1 and 0.5, e.g. about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9 or 1%. The organic liquid may be a solvent for the monomer or monomers. It may be a solvent for all of the monomers if more than one is used. It may be capable of dissolving the monomers in the proportions used in the dispersed phase of the dispersion. Thus the solubility of the monomer, or each of the monomers, or the mixture of monomers, may be between about 5 and about 75% w/w or v/v, as described above. The solubilities and swelling described above may be determined at the reaction temperature used or at room temperature. Thus the organic liquid may be selected so as to be a solvent for the monomer(s) and a non-solvent and non-swelling liquid for a polymer formed by polymerisation (or copolymerisation) of the monomer(s).

The monomer may be polymerisable, or copolymerisable with the further is monomers if present, by means of thermally initiated polymerisation. The monomer may be polymerisable, or copolymerisable with the further monomers if present, by means of radiation initiated polymerisation. If thermal polymerisation is to be used, the discontinuous organic phase may comprise a thermal initiator. These are well known to those skilled in the art. They include azo initiators, peroxides, peroxyesters and hydroperoxides, and may be selected to have the desired half-life at the reaction temperature used. Suitable initiators include 2,2′-azobis(isobutyronitrile), 4,4′-azobis(4-cyanopentanoic acid), 2,2′-azobis-(2,4-dimethylvaleronitrile), t-butyl hydroperoxide, cumene hydroperoxide, benzoyl peroxide and lauroyl peroxide, or mixtures thereof. If UV initiation is to be used, the discontinuous organic phase may comprise a photoinitiator. These also are well known in the art. They include benzoin ethers, benzil ketals, α-dialkoxyacetophenones, α-hydroxyalkylphenones, α-aminoalkylphenones, acylphosphine oxides, benzophenones/amines, thioxanthones/amines, titanocenes (for visible light initiation) etc. The thermal initiator or photoinitiator, if present, may be present in the discontinuous phase in a concentration of between about 0.1 and 5 wt %, depending on the nature of the initiator, or between about 0.1 and 2, 0.1 and 1, 0.1 and 0.5, 0.5 and 5, 1 and 5, 2 and 5, 0.5 and 2, 1 and 2, 1 and 3 or 1.5 and 2.5%, e.g. about 0.1, 0.2, 0.3, 0.4, 0.5, 0.6, 0.7, 0.8, 0.9, 1, 1.5, 2, 2.5, 3, 3.5, 4, 4.5 or 5%, or it may be less than 0.1% or greater than 5%. The step of providing the dispersion may comprise preparing the dispersion. This may comprise combining a mixture (optionally a solution) of the stabiliser in water or some aqueous liquid in the desired ratio (as described above) with a mixture, optionally solution, of the monomer (and optionally further monomers), intiator (if used), and the organic liquid. Other orders of addition may readily be appreciated by those skilled in the art. The dispersion or any of its components may be degassed (or deoxygenated) and/or agitated during and/or after preparation. The degassing or deoxygenation may comprise sparging with nitrogen, argon, helium or other suitable non-oxygen containing gas, or it may comprise one or more (e.g. 1, 2, 3, 4 or 5) freeze-pump-thaw cycles, or may comprise both of the above. The agitation may comprise swirling, sonicating, shaking, stirring or otherwise agitating. The degassing (or deoxygenation) and/or agitation may be sufficient to reduce the oxygen concentration in the dispersion sufficiently that it does not inhibit polymerisation of the monomer and optionally the further monomers.

Prior to polymerising the monomer, the discontinuous organic phase may not contain a polymer. It may not contain a solid polymer. It may not contain polymeric microparticles. In earlier work, a polymer was swelled with a monomer and a solvent in the discontinuous phase of a dispersion. However in those experiments it was shown that the presence of a polymer was essential to the formation of hollow microparticles. By contrast, in the present work, no polymer may be present. The inventors surprisingly find that by use of the correct conditions, hollow microparticles may be obtained without adding a preformed polymer to the dispersion. It will be understood that the absence of polymer in the discontinuous organic phase does not preclude very low concentrations of polymeric stabiliser that may transfer from the continuous aqueous phase. Thus prior to polymerisation (i.e. prior to step b of the first aspect) the discontinuous organic phase may contain no hydrophobic polymer. It should also be recognised that a limited amount of adventitious polymerisation of monomers may in some cases occur prior to the polymerisation step, and thus very low concentrations of hydrophobic polymer may also be present (commonly low molecular weight material, e.g. oligomers). This should not be considered to take the process outside the scope of the present invention. Thus the discontinuous organic phase may contain no deliberately added polymer prior to polymerisation (i.e. prior to step b of the first aspect) That is, any polymer present in the discontinuous organic phase prior to polymerisation (i.e. prior to step b of the first aspect) may be not deliberately added to the discontinuous organic phase. In particular the discontinuous organic phase may contain no deliberately added hydrophobic polymer prior to polymerisation (i.e. prior to step b of the first aspect). The discontinuous organic phase may contain substantially no polymer, in particular substantially no hydrophobic polymer, prior to polymerisation (i.e. prior to step b of the first aspect).

The absence of the requirement to add polymer to the system prior to polymerisation of the monomer(s) may serve to simplify particle size control, as well understood principles of emulsion formation and droplet size control may be used to control the particle size of the resulting microparticles.

Prior to step b) of the first aspect, the discontinuous organic phase may consist essentially of a monomer having two or more polymerisable groups per molecule and an organic liquid. Prior to step b) of the first aspect, the discontinuous organic phase may consist essentially of a monomer having two or more polymerisable groups per molecule, an organic liquid and a polymerisation initiator and/or photosensitiser. Prior to step b) of the first aspect, the discontinuous organic phase may consist essentially of a monomer having two or more polymerisable groups per molecule, a second monomer (and optionally further monomers) which is (are) copolymerisable with the monomer having two or more polymerisable groups per molecule, an organic liquid and a polymerisation initiator and/or photosensitiser.

Prior to step b) of the first aspect, the discontinuous organic phase may consist of a monomer having two or more polymerisable groups per molecule and an organic liquid. Prior to step b) of the first aspect, the discontinuous organic phase may consist of a monomer having two or more polymerisable groups per molecule, an organic liquid and a polymerisation initiator and/or photosensitiser. Prior to step b) of the first aspect, the discontinuous organic phase may consist of a monomer having two or more polymerisable groups per molecule, a second monomer (and optionally further monomers) which is (are) copolymerisable with the monomer having two or more polymerisable groups per molecule, an organic liquid and a polymerisation initiator and/or photo sensitiser.

Step b) of the process comprises polymerising the monomer in the dispersion to form a polymer in the form of hollow microparticles. As noted above, the polymerisation may be initiated thermally, photochemically or in some other fashion. The polymerisation should be initiated in the discontinuous organic phase of the dispersion. Thus step b) may comprise initiating polymerisation, optionally copolymerisation. It may comprise heating the dispersion. The heating may be accomplished by use of a heater, a microwave generator, a heating bath or some other manner. The heating may be to the desired reaction temperature, which will depend on the nature of the thermal initiator. It may be between about 50 and about 100° C., or between about 60 and 100, 70 and 100, 80 and 100, 90 and 100, 50 and 90, 50 and 80, 50 and 70, 50 and 60, 60 and 90, 60 and 75 or 75 and 90, e.g. about 50, 55, 60, 65, 70, 75, 80, 85, 90, 95 or 100° C. The heating may be to reflux temperature. The heating may be to a temperature such that the half-life of the initiator is between about 5 minutes and about 20 hours, or between about 5 minutes and 10 hours, 5 minutes and 5 hours, 5 minutes and 2 hours, 5 minutes and 1 hour, 5 and 45 minutes, 5 and 30 minutes, 5 and 15 minutes, 5 and 10 minutes, 10 minutes and 2 hours, 30 minutes and 2 hours, 1 and 2 hours, 1 and 20 hours, 1 and 10 hours, 10 and 20 hours, 1 and 5 hours, 2 and 10 hours, 1.5 and 2 hours, 10 minutes and 1 hour, 10 and 30 minutes or 10 and 20 minutes, e.g. about 5, 10, 15, 20, 25, 30, 35, 40, 45, 50 or 55 minutes, or about 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 11, 12, 13, 14, 15, 16, 17, 18, 19 or 20 hours, or more than 20 hours. If the initiation is photoinitiation, the wavelength of the irradiation used may be appropriate to the photoinitiator used. In the case of other radiation sources e.g. gamma radiation, no initiator may be required.

The polymerisation time should be sufficient to achieve a conversion of at least about 80%, or at least about 85, 90 or 95%, or between about 80 and 100, 85 and 100, 90 and 100, 95 and 100, 99 and 100, 80 and 95, 80 and 90, 80 and 85, 85 and 90 or 90 and 99%, e.g. about 80, 85, 90, 91, 92, 93, 94, 95, 96, 97, 98, 99 or 100%. The polymerisation time will depend, amongst other factors, on the nature of the monomer(s), the conditions used and the nature of the initiator. Commonly the polymerisation time will be between about 1 and 24 hours, or between about 6 and 24, 12 and 24, 18 and 24, 1 and 12, 1 and 6, 1 and 3, 6 and 18, 12 and 18 or 18 and 22 hours, e.g. about 1, 2, 3, 4, 5, 6, 7, 8, 9, 10, 11, 12, 13, 14, 15, 16, 17, 18, 19, 20, 21, 22, 23 or 24 hours, although it may be less than 1 hour or greater than 24 hours in certain circumstances. For example the polymerisation time for rapid polymerisation systems may be between about 1 and about 60 minutes, or between about 1 and 30, 1 and 20, 1 and 10, 1 and 5, 5 and 60, 10 and 60, 30 and 60 or 10 and 30 minutes, e.g. about 1, 2, 3, 4, 5, 6, 7, 8, 910, 15, 20, 25, 30, 35, 40, 45, 50, 55 or 60 minutes. The time may be sufficient for the degree of conversion of the monomer to polymer to be between about 10 and 100%, or between about 20 and 100, 50 and 100, 60 and 100, 70 and 100, 80 and 100, 90 and 100, 10 and 90, 10 and 80, 10 and 70, 10 and 60, and 50, 25 and 90, 50 and 90, 50 and 80, 60 and 80, 70 and 90, 70 and 80 or 80 and 90%, e.g. about 10, 20, 30, 40, 50, 55, 60, 65, 70, 75, 80, 85, 90, 95 or 100%.

Suspension polymerization is usually conducted in an oil in water system. However, inverse suspension polymerization can be carried out using water in oil systems. This may be suitable for polymerisation of hydrophilic monomers. Suspension polymerization is more commonly applied to hydrophobic monomers. However, it is in some cases desirable to produce hollow microparticles whose shells comprise hydrophilic polymers, which are capable of swelling in water. In order to achieve this, the polymerization may be conducted using protected monomers, which are hydrophobic, but may be rendered hydrophilic upon deprotection (e.g hydrolysis). In one example, a methacrylate monomer is used as a comonomer in the polymerisation, and the resulting copolymer may be hydrolyzed to provide a copolymer comprising methacrylic acid derived monomer units. The resulting particles are not only hydrophilic, but also stimuli-responsive. Depending on the pH of the medium in which the resulting hollow microparticles are located, the carboxylate units are either protonated (not very hydrophilic) or charged (particles will swell substantially in water)

In a complementary approach, the crosslinker is degradable, but not by alteration of pH. For example, thiol containing compounds, as are present in peptides and proteins, may be used cleave a disulfide bridge present in a copolymer, resulting in degradation of the polymer network. Thus if a crosslinking monomer containing a disulfide linkage (e.g. bis(2-methacryloyloxyethyl)disulfide) is used to make the shell of the hollow microparticles, the resulting microparticles may be degraded by exposure to a thiol containing species, but not by exposure to a hydrolytic species.

In some cases, following formation of the hollow microparticles, a subsequent step may be conducted to cleave, e.g. hydrolyse, a bond in the microparticles to render the surface of the microparticles hydrophilic. In one example, the crosslinking monomer contains a non-hydrolysable crosslinking group, e.g. a disulfide link, and a second monomer contains a hydrolysable group, e.g. an ester. In this case, hydrolysis of the ester to form a carboxylate group may be effected without cleaving the crosslinking groups of the polymer, i.e. without degrading the copolymer. Hydrolysis may be conducted using an aqueous acid (e.g. HCl, HF, HBr, HNO₃, H₂SO₄ or some other strong acid). The aqueous acid may be dispersed or dissolved in an organic solvent, e.g. a water miscible organic solvent such as acetone, dioxane, DMF, DMSO etc. The aqueous acid may be between about 5 and about 50% in the water, or about 5 to 30, 5 to 20, 10 to 50, 20 to 50 or 5 to 15% (w/v), e.g. about 5, 10, 15, 20, 30, 40 or 50%. Alternatively the hydrolysis may be conducted in an organic soluble strong acid dissolved in an organic solvent. Suitable organic acids include trifluoroacetic acid. Suitable solvents include chloroform, dichloromethane, diethyl ether etc. Hydrolysis may also be conducted using other reagents, for example bases (e.g. aqueous bases), enzymes etc. The hydrolysis may be conducted at about 20 to about 100° C. (depending on the boiling point of the solvent) or about 20 to 80, 20 to 60, 20 to 40, 40 to 100, 60 to 100 or 40 to 80° C., e.g. about 20, 30, 40, 50, 60, 70, 80, 90 or 100° C. It may take between about 6 and about 60 hours, or about 6 and 48, 8 and 24, 6 and 12, 12 and 60, 24 and 60, 36 and 60, 24 and 48, 12 and 36 or 36 and 60 hours, e.g. 6, 12, 18, 24, 36, 48 or 60 hours. It may be conducted for sufficient time to achieve the desired degree of hydrolysis at the reaction temperature used. The degree of hydrolysis may be about 10 to about 100% on a weight basis, or about 20 to 100, 50 to 100, 80 to 100, 10 to 50, 10 to 30, 20 to 90, 50 to 90 or 20 to 50%, e.g. about 10, 20, 30, 40, 50, 60, 70, 80, 90 or 100%.

Following step (b), and optionally following hydrolysis, if performed, the process may in some cases comprise one or more of the following steps:
separating the microparticles from the continuous aqueous phase
washing the microparticles
drying the microparticles, and
removing the organic liquid from the microparticles and/or, if the organic liquid or a component thereof polymerises during step (b), removing a polymer formed from the organic liquid or component thereof from the microparticles.

The step of separating may comprise filtering, microfiltering, centrifuging, ultracentrifuging, settling, decanting, skimming etc. The step of washing may use one or more solvents including aqueous solvents (water, saline etc.), organic solvents (polar or apolar solvents) or a combination of these. The washing may be repeated, and may be repeated using the same or different solvents. The step of drying may comprise exposing the microparticles to a stream of gas (e.g. air, nitrogen, carbon dioxide etc.), optionally heated gas. The step of drying may comprise heating the microparticles (e.g. to a temperature between about 5 and 100° C.). The step of drying may comprise applying an at least partial vacuum (e.g. less than about 10, 5, 2 or 1 mBar). The step of drying may comprise a combination of these. The step of removing the organic liquid from the microparticles may comprise exposing the microparticles to a stream of gas (e.g. air, nitrogen, carbon dioxide etc.), optionally heated gas. The step of removing the organic liquid from the microparticles may comprise heating the microparticles (e.g. to a temperature between about 5 and 100° C.). The step of removing the organic liquid from the microparticles may comprise applying an at least partial vacuum (e.g. less than about 10, 5, 2 or 1 mBar. The step of removing the organic liquid from the microparticles may comprise a combination of these. If the organic liquid or a component thereof polymerises during step (b), the step of removing the polymer so formed may comprise washing the microparticles with a suitable solvent, optionally a volatile solvent. The steps of drying and removing the organic liquid may be conducted concurrently. They may be conducted separately. If the microparticles are loaded with a substance, this may be conducted before any of the above steps. It may be conducted during any of the above steps. It may be conducted after any of the above steps.

The present invention encompasses microparticles per se. The microparticles of the present invention may be spherical or may be ovoid, oblate spherical, elongated spherical, pseudospherical or may be polyhedral (having between about 8 and about 50 sides), optionally regular polyhedral or other shape. They may be hollow microspheres. They may be microspheres that are loaded with a desired substance within a cavity in each microsphere. They may be hollow micropseudospheres. They may be micropseudospheres that are loaded with a desired substance within a cavity in each micropseudosphere. They may have a mean diameter between about 0.1 microns and about 2 mm or between about 0.1 microns and 1 mm, 0.1 and 500 microns, 0.1 and 200 microns, 0.1 and 100 microns, 0.1 and 50 microns, 0.1 and 10 microns, 0.1 and 5 microns, 0.1 and 1 micron, 1 micron and 2 mm, 10 microns and 2 mm, 50 microns and 2 mm, 100 microns and 2 mm, 200 microns and 2 mm, 500 microns and 2 mm, 1 and 2 mm, 1 and 500 microns, 1 and 200 microns, 1 and 100 microns, 1 and 50 microns, 1 and 20 microns, 1 and 10 microns, 10 and 500 microns, 50 and 500 microns, 100 and 500 microns, 200 and 500 microns, 10 and 200 microns or 10 and 100 microns. They may have a mean diameter of e.g. about 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 600, 700, 800 or 900 microns. They may have a mean diameter of about 1, 1.1, 1.2, 1.3, 1.4, 1.5, 1.6, 1.7, 1.8, 1.9 or 2 mm. Thus the present specification describes the synthesis of hollow microparticles (microcapsules) where the size of the capsule is given by the empirical relationship:

$$d = k \frac{{D}_{v} {Rv}_{d} ɛ}{{D}_{s} {Nv}_{m} {C}_{s}}$$

in which:
d=average particle size,
k=parameters representing reactor design, type of stirrer, self stabilization etc . . . ,
Dᵥ=diameter of vessel,
Dₛ=diameter of stirrer,
R=volume ratio of the droplet phase to the suspension medium,
N=stirring speed,
v_{d}=viscosity of the droplet phase,
vₘ=viscosity of the suspension medium,
ε=interfacial tension between the two immiscible phases,
Cₛ=stabilizer concentration.

The microparticles of the present invention are hollow, i.e. they have a cavity in the interior thereof. The cavity may be spherical, or may be ovoid, oblate spherical, elongated spherical, pseudospherical, pseudoovoid, cubical, pseudocubical or may be polyhedral (having between about 8 and about 50 sides), irregular polyhedral or regular polyhedral. Each microparticle may have a single cavity. The cavity may represent between about 5 and about 75% of the volume of the microparticle or between about 5 and 60, 5 and 50, 5 and 40, 5 and 30, 5 and 25, 5 and 10, 10 and 75, 25 and 75, 50 and 75, 20 and 70, 30 and 60, 40 and 60 or 45 and 55, e.g. about 5, 10, 15, 20, 25, 30, 35, 40, 45, 50, 55, 60, 65, 70 or 75%. The cavity may have a diameter of between about 0.05 and 1500 microns, depending in part on the diameter of the particle in which the cavity is located. The cavity may have a diameter between about 0.1 and 1500, 1 and 1500, 10 and 1500, 50 and 1500, 100 and 1500, 500 and 1500, 1000 and 1500, 0.05 and 1000, 0.05 and 500, 0.05 and 100, 0.05 and 50, 0.05 and 10, 0.05 and 5, 0.05 and 1, 0.05 and 0.1, 1 and 1000, 10 and 1000, 100 and 1000, 1 and 500, 1 and 100, 1 and 10, 10 and 1000, 10 and 500, 10 and 100 or 100 and 1000 microns, e.g. about 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1100, 1200, 1300, 1400 or 1500 microns. The cavity in a hollow microparticle may have a diameter of between about 1 and about 70% of the diameter of the microparticle. The cavity may have a diameter of between about 5 and 60, 5 and 50, 5 and 40, 5 and 30, 5 and 25, 5 and 10, 10 and 75, 25 and 75, 50 and 75, 20 and 70, 30 and 60, 40 and 60 or 45 and 55% of the diameter of the microparticle, e.g. about 5, 10, 15, 20, 25, 30, 35, 40, 45, 50, 55, 60, 65, 70 or 75% of the diameter of the microparticle. The hollow microparticle may be a hollow spherical microparticle. The hollow microparticle may be a hollow cubical, rhombohedral, or irregular shaped microparticle. The cavity is surrounded by a polymeric shell. It should be understood that the term “hollow” in this context refers to a structure wherein the microparticle comprises a polymeric shell surrounding an inner region (cavity or core) that is not polymeric. The polymeric shell or wall may have a thickness of between about 0.02 and about 500 microns (depending in part on the diameter of the microparticle) or between about 0.05 and 500, 0.1 and 500, 0.2 and 500, 0.5 and 500, 1 and 500, 5 and 500, and 500, 20 and 500, 50 and 500, 100 and 500, 200 and 500, 300 and 500, 400 and 500, 0.02 and 200, 0.02 and 100, 0.02 and 50, 0.02 and 20, 0.02 and 10, 0.02 and 5, 0.02 and 2, 0.02 and 1, 0.02 and 0.5, 0.02 and 2, 0.02 and 1, 1 and 500, 1 and 200, 1 and 100, 1 and 50, 1 and 10, 10 and 500, 50 and 500, 100 and 500 or 10 and 100 microns, e.g. about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 65, 80, 85, 90, 95, 100, 150, 200, 250, 300, 350, 400, 45 or 500 microns. The thickness may be between about 1 and about 45% of the microparticle diameter, or between about 1 and 40, 1 and 35, 1 and 30, 1 and 25, 1 and 20, 1 and 15, 1 and 10, 1 and 5, 5 and 45, 10 and 45, 20 and 45, 30 is and 45, 5 and 40, 5 and 20, 10 and 30 or 10 and 20%, e.g. about 1, 2, 3, 4, 5, 6, 7, 8, 9, 10, 15, 20, 25, 30, 35, 40 or 45%. The inner region or cavity may contain various substances, including the organic liquid, other solvents, an encapsulated substance etc.

The polymeric shell of the microparticles may comprise (or consist of) a crosslinked polymer. It may comprise a crosslinked acrylic polymer. It may comprise a crosslinked acrylic copolymer. It may comprise a crosslinked acrylate/methacrylate copolymer. The crosslinks of the polymer may be derived from difunctional, trifunctional or polyfunctional crosslinking monomers. The crosslinking monomers may be acrylate esters or methacrylate esters. They may be di-, tri- or poly-esters of a glycol or of a glycol oligomer (e.g. ethylene glycol dimethacrylate, diethylene glycol dimethacrylate, ethylene glycol diacrylate etc.). Examples of suitable polymers include poly(ethyleneglycol dimethacrylate-co-vinyl neodecanoate), poly(ethyleneglycol dimethacryate), poly(ethyleneglycol dimethacrylate-co-methyl methacrylate), poly(ethyleneglycol dimethacrylate-co-t-butyl methacrylate), poly(ethyleneglycol dimethacrylate-co-methacrylic acid), poly(ethyleneglycol dimethacrylate-co-t-butyl methacrylate-co-bis(2-methacryloyloxyethyl)disulfide), poly(t-butyl methacrylate-co-bis(2-methacryloyloxyethyl)disulfide) and poly(methyl methacrylate-co-bis(2-methacryloyloxyethyl)disulfide).

The microparticles of the present invention may be formed by the process of the invention.

The empty hollow microparticles can be loaded with a range of compounds including metal ions and complexes, various polymers or nanoparticles or combinations of these. The loading may be carried out during the synthesis. The loading may be carried out as post treatment.

Thus in some embodiments the present invention discloses hollow microparticles comprising a polymeric shell surrounding a hollow core, said hollow core containing a substance and said polymeric shell being at least partially degradable so as to release the substance from the core. The polymeric shell may be degradable hydrolytically. It may be degradable by means of a thiol. It may be degradable reductively. It may be degradable enzymatically. It may be biodegradable. It may be degradable oxidatively. It may be degradable by means of reagent which cleaves a crosslink bond in said polymeric shell. The polymeric shell may comprise units derived from a crosslinking monomer. The polymeric shell may comprise units derived from a crosslinking monomer and units derived from a non-crosslinking monomer. The ratio of units derived from the crosslinking monomer to units derived from the non-crosslinking monomer may be between about 1:20 and about 20:1 or about 1:10 and 10:1, 1:5 and 5:1, 1:2 and 2:1, 1:1.1 and 1.1 to 1, 1:20 and 1:1, 1:10 and 1:1, 1:5 and 1:1, 1:2 and 1:1, 2:1 and 1:1, 5:1 and 1:1, 10:1 and 1:1 or 20:1 and 1:1, e.g. about 20:1, 10:1, 5:1, 2:1, 1.5:1, 1.1:1, 1:1, 1:1.1, 1:1.5, 1:2, 1:5, 1:10 or 1:20. Alternatively the polymeric shell may comprise no units derived from a non-crosslinking monomer. The above ratios may be on a mole basis. The polymeric shell may be hydrophobic. It may be chemically convertible (e.g. by hydrolysis) to a hydrophilic polymeric shell. It may be chemically convertible (e.g. by hydrolysis) to a hydrophilic shell by a process which does not cleave crosslinks in said shell. It may be chemically convertible (e.g. by hydrolysis) to a hydrophilic shell by a process which does not cleave crosslinks in said shell, said crosslinks being subsequently cleavable so as to release a substance from the core of the microparticles. The polymeric shell may be hydrophilic. The polymeric shell may be hydrophilic and may comprise crosslinks which are cleavable, optionally be a non-hydrolytic process, in order to release a substance from the core of the microparticles.

The release of the substance in the core from the microparticles may be rapid or it may be slow. The time taken to release 50% of the substance in the core of the particles from the microparticles may be between about 1 minute and about 1 year, or may be about 1 minute to 1 month, 1 minute to 1 week, 1 minute to 1 day, 1 minute to 1 hour, 1 to 30 minutes, 1 to 10 minutes, 1 hour to 1 year, 1 day to 1 year, 1 week to 1 year, 1 month to 1 year, 6 months to 1 year, 1 hour to 1 day, 1 day to 1 week, 1 week to 1 month, 1 to 6 months, 1 to 12 hours, 1 to 2 days, 1 to 2 weeks or 1 to 2 months, e.g. about 1, 2, 3, 4, 5, 10, 20, 30, 40 or 50 minutes, 1, 2, 3, 4, 5, 6, 12 or 18 hours, 1, 2, 3, 4, 5 or 6 days, 1, 2 or 3 weeks or 1, 2, 3, 4, 5, 6, 7, 8, 9, 10, 11 or 12 months. The release rate will depend on the conditions to which the microparticles are exposed. Those conditions (temperature, pH, concentration of reagents etc.) may be such as to obtain the desired release rate, as described above. In some embodiments of the invention, the substance in the core of the microparticles is not released.

The process may additionally comprise the step of loading a substance into the interior region of the hollow microparticles to form loaded microparticles. The step of loading the substance may comprise infusing the substance into the microparticles. This may be achieved by suspending the microparticles in a solvent containing the substance in solution. The solvent may be an organic solvent. It may be a polar solvent. The step of loading may be conducted at room temperature. It may be conducted at elevated temperature. It may be conducted at any suitable temperature up to and including the boiling point of the solvent. It may be conducted for sufficient time to achieve the desired loading. The time required may be between about 5 minutes and about 30 days or more, or between about 5 minutes and 20 days, 5 minutes and 10 days, 5 minutes and 5 days, 5 minutes and 2 days, 5 minutes and 1 day, 5 minutes and 18 hours, 5 minutes and 12 hours, 5 minutes and 6 hours, 5 minutes and 3 hours, 5 minutes and 2 hours, 5 minutes and 1 hour, 5 and 30 minutes, 5 and 20 minutes, 5 and 10 minutes, 30 minutes and 30 days, 1 hour and 30 days, 2 hours and 30 days, 6 hours and 30 days, 12 hours and 30 days, 1 and 30 days, 5 and 30 days, 10 and 30 days, 20 and 30 days, 1 hour and 20 days, 1 hour and 2 days, 1 hour and 1 day, 1 and 6 hours, 1 and 20 days, 1 and 10 days, 1 and 5 days or 5 and 10 days, e.g. about 5, 10, 15, 20, 25, 30, 35, 40, 45, 50 or 55 minutes, 1, 2, 3, 4, 5, 6, 12, 15, 18 or 21 hours or 1, 2, 3, 4, 5, 6, 7, 8, 9, 10, 15, 20, 25 or 30 days.

The substance may be a drug. It may be usable in the treatment of cancer. It may be capable of generating a β-emitter when subjected to neutron bombardment. It may for example be an yttrium salt e.g. yttrium nitrate. In the event that the substance when bombarded by neutrons generates a β-emitter, this is useful since the particles loaded with a non-radioactive substance may be prepared, stored and transported in safety, and the encapsulated substance may be converted to a β-emitter shortly prior to use (e.g. administration to a patient), thereby reducing the possibility of exposure of users to dangerous radioactivity.

In an alternative, the substance to be loaded into the hollow microparticles may be included in the discontinuous organic phase of the dispersion in step (a) of the process, whereby it is incorporated into the microparticles, optionally into the cavity of the microparticles, during the formation thereof. The substance may be soluble in the organic liquid (or in the organic phase). It may be insoluble therein. It may be dispersible in the organic liquid (or in the organic phase). It may be present in the organic phase in any suitable concentration e.g. between about 0.01 and about 10%, or between about 0.01 and 5, 0.01 and 2, 0.01 and 1, 0.01 and 0.5, 0.01 and 0.2, 0.01 and 0.1, 0.01 and 0.05, 0.1 and 10, 0.5 and 10, 1 and 10, 5 and 10, 0.1 and 1, 1 and 5, 0.1 and 0.5 or 0.5 and 1% w/w or w/v, e.g. about 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.6, 0.7, 0.8, 0.9, 1, 1.5, 2, 2.5, 3, 3.5, 4, 4.5, 5, 6, 7, 8, 9 or 10%. A suitable concentration may depend on factors such as cost, solubility, required amount to be encapsulated etc. In this case, the substance should be capable of withstanding the conditions used in the polymerisation (e.g. temperature, radiation etc.) without substantial degradation.

The invention also provides a method of treating a condition in a patient comprising administering to said patient a therapeutically effective quantity of microparticles according to the invention, wherein a substance indicated for treatment of said condition is located in the interior region of the microparticles. The patient may be a human. It may be a non-human animal, e.g. a non-human mammal, fish, bird, vertebrate or invertebrate. The administering may be orally, or by inhalation, or by injection. The injection may be intravenous, intramuscular or subdermal. The microparticles of the present invention may have no surfactant therein or thereon, as it is possible to make the microparticles without the use of surfactants. This may be an advantage in applications in which the microparticles are used internally to a patient, as surfactants can generate adverse reactions in a patient.

The method may be a method of treating cancer in a patient, comprising:
exposing a therapeutic quantity of hollow microparticles according to the invention to neutrons from a neutron source, wherein a substance in the interior region of the hollow microparticles is such that the exposure generates a β-emitter; and
administering the therapeutic quantity of hollow microparticles to the patient.

The hollow microparticles according to the invention may be used for the manufacture of a medicament for the treatment of cancer, or for the treatment of some other condition. They may therefore be combined with one or more suitable carriers and/or adjuvants for administration to a patient. Suitable carriers include water (sterile water, USP water, BP water), saline, Ringer's solution and other carriers known to those skilled in the art. When so used, the microparticles should contain within the core a substance indicated for the treatment of the cancer or other condition as appropriate. The substance may be a drug, or a prodrug.

Many applications for hollow microparticles require the synthesis of stimuli-responsive or degradable systems. Therefore, the use of crosslinkers which can potentially degrade over time has been demonstrated. Many ester-type crosslinker are known to undergo slow hydrolysis under physiological conditions or simply in an aqueous environment. Ester hydrolysis is typically known to take place at a rather slow rate.

Therefore, other crosslinkers such as anhydrides, orthoester and acetals may also be used in order to broaden the application of these microparticles to areas where an almost instant decomposition of the microparticles is required. As noted earlier, non-hydrolytically degradable crosslinking groups may also be introduced into the microparticles. For example disulfide crosslink groups are not readily hydrolysed, but may be degraded by exposure to a thiol, such as those present in proteins and peptides.

The invention also provides a medicament for treating a condition in a patient, said medicament comprising microparticles according to the present invention, said microparticles comprising a substance indicated in treatment of said condition. The medicament may be in the form of a capsule containing said microparticles, or it may be in the form of a suspension of said microparticles in a suitable carrier. It may be suitable for oral administration. It may be suitable for injection.

### BRIEF DESCRIPTION OF THE DRAWINGS

Preferred embodiments of the present invention will now be described, by way of example only, with reference to the accompanying drawings wherein:

FIG. 1 is a scheme illustrating synthesis of a three layered particle system with the monomer (VND), crosslinker (EGDMA) and co-monomer (PEGMA);

FIG. 2 shows micrographs (left hand micrograph—optical micrograph; centre and right hand micrograph—scanning electron micrographs) of hollow microparticles prepared according to the present invention;

FIG. 3 shows TGA analysis of Y(NO₃)₃ loaded hollow particles with different solvents used for drug loading;

FIG. 4 is a diagram illustrating the incorporation of drugs into hollow microspheres;

FIG. 5 is a diagram illustrating formation of poly(EGDMA) hollow spheres with BuAc as solvent by suspension polymerization;

FIG. 6 shows electron micrographs of the spheres made using 50% and 95% EGDMA;

FIG. 7 is a diagram showing formation of poly (MMA-co-EGDMA) hollow spheres;

FIG. 8 is a diagram showing formation of poly (tBuMA-co-EGDMA) hollow spheres;

FIG. 9 shows micrographs of spheres produced using 10% EGDMA-10% tBuMA and 20% EGDMA-20% tBuMA;

FIG. 10 is a diagram showing conversion of hydrophobic into hydrophilic hollow microspheres;

FIG. 11 is a diagram showing formation of peptide-induced degradable hollow spheres containing disulfide crosslinks;

FIG. 12 is a diagram showing formation of pH-sensitive hollow spheres; and

FIG. 13 is a diagram showing a different approach to formation of pH-sensitive hollow spheres from that of FIG. 12.

### DETAILED DESCRIPTION OF PREFERRED EMBODIMENTS

Core-shell microspheres may be prepared according to the process of the present invention via the suspension polymerization technique. The following provides an example of the process of the present invention.

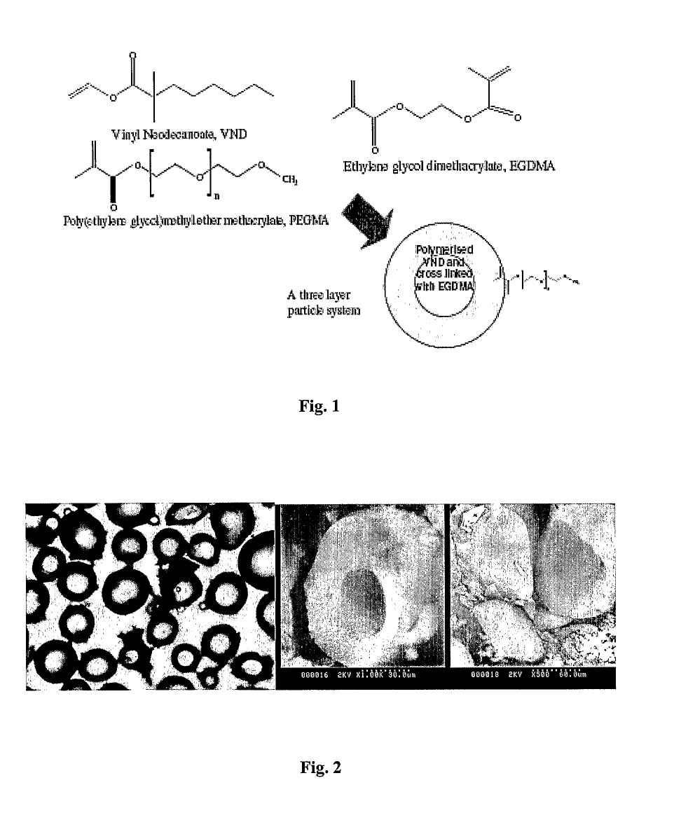

Due to the hydrophilic nature of the blood stream, poly(ethylene glycol)methacrylate (PEGMA) was introduced as a co-monomer in a one step reaction to yield microparticles with a hydrophilic shell. The particle core comprised polymerized vinyl neodecanoate, VND and ethylene glycol dimethacrylate, EGDMA as a crosslinker, (FIG. 1).

The position at which PEGMA was polymerized was determined by an interfacial tension test and a stability test. Results show that PEGMA is located at the interface of the dispersed and the continuous phase. Moreover, PEGMA was shown to have an affinity for the continuous aqueous phase. The results from these two tests revealed that PEGMA did polymerize into the shell surface of the microparticles formed. Confirmations on the reacted poly(ethylene glycol)methacrylate (PEGMA) were conducted with the assistance of ¹H-NMR analysis. The size distribution of these core-shell particles were also analysed using Scanning Electron Microscope (SEM) and Coulter® dynamic light scattering machine.

The microparticles were shown to be hollow when these core-shell particles were exposed to hexane to remove the inner core which comprised of VND. It was concluded that the particles had a three layered particle structure which contain VND, EGDMA and PEGMA.

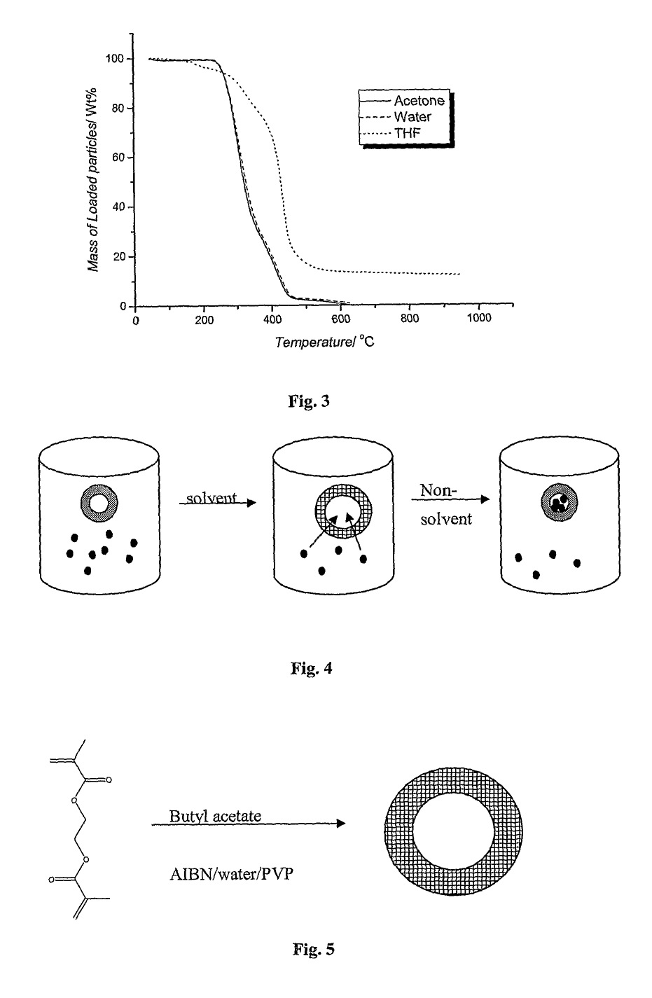

Empty particles were subsequently synthesized using butyl acetate (BuAc) as a template (FIG. 2). The first picture of FIG. 2 is an optical micrograph, and shows the hollow structure of the microparticles while they are still intact. The dark corona and bright core in the optical micrograph indicates the hollow structure. The second and third pictures of FIG. 2 are electron micrographs, which confirm the hollow structure of the microparticles. Encapsulation of these empty particles with yttrium nitrate, Y(NO₃)₃, allows the latter to be activated by neutron bombardment to form β-emitters. They may then be injected into the vicinity of cancer cells to impart a large localized dose of β-radiation. Y(NO₃)₃ loading was conducted using tetrahydrofuran (THF) as a solvent and analysed by examining the weight lost via thermogravimetric analysis (TGA). A significant amount of Y(NO₃)₃ was found to be encapsulated into the particles, (FIG. 3).

Thus hollow microparticles were successfully synthesized via the suspension polymerization method. In addition, the encapsulation of yttrium nitrate into the hollow microspheres was conducted by diffusion of Y(NO₃)₃ dissolved in THF.

Drugs may be loaded into the hollow microspheres by stirring the microspheres in the presence of a concentrated, e.g. saturated, solution of the drug. The solvent depends on the drug used and the polarity of the microsphere.

The solvent for drug loading should
dissolve the drug in high concentrations; and
be able to swell the particles significantly.

In a typical procedure, hollow microspheres, drug and solvent are carefully stirred while allowing the slow evaporation of the solvent to maintain a sufficient concentration gradient between solution and the interior of the hollow sphere. Drug loading is usually complete within 2 hours, depending on the system. The microspheres are then centrifuged and the solvent is removed. The loaded particles are then washed, preferably with a non-solvent for the polymer, to achieve the collapse (i.e. deswelling) of the hollow microsphere and the encapsulation of the drug.

The non-solvent should:
be able to collapse (i.e. deswell) the swollen microsphere quickly;
be a reasonable solvent for the drug to remove excess drug deposited on the surface.

A diagram illustrating the incorporation of drugs into hollow microspheres is shown in FIG. 4.

### Abbreviation Used in Examples Below are:

### BuAc: Butyl Acetate

EGDMA: Ethylene glycol dimethacrylate
MMA: Methyl methacrylate
PVP: poly(N-vinyl pyrrolidone)
tBuMA: tert-Butyl Methacrylate

### Example 1

The reactor was a 250 ml wide mouth flask modified to include four 10 mm radial baffles with removable 5 neck lid. It was equipped with an overhead stirrer, 2 four bladed 40 mm turbine impeller, a condenser and an oil bath. A mixture of 1.05 g PVP and 199.5 g water was added to the reactor and purged with nitrogen with the aid of a sonicator. After the degassing and sonication step, a mixture of ethylenegycol dimethacrylate EGDMA (2.0 g), vinyl neodecanoate (8.0 g), poly(ethyleneglycol) methacrylate (1.0 g), initiator (AIBN: 0.2 g, 0.095 wt %, 2 wt % of oil phase) and butyl acetate (5.0 g) as a non-solvent was introduced to the reaction flask and degassing was continued with slow stirring at 200 rpm for another 30 minutes. Reaction was started by ramping the temperature (20° C.) from room temperature to 70° C. in 1 hour. After a reaction time of 20 hours the microspheres were filtered off and washed with water and acetone. To remove the core of the particle the particles were suspended in hexane, filtered and washed thoroughly with hexane.

### Example 2

The reactor was a 250 ml wide mouth flask modified to include four 10 mm radial baffles with removable 5 neck lid. It was equipped with an overhead stirrer, 2 four bladed 40 mm turbine impeller, a condenser and an oil bath. A mixture of 1.05 g PVP and 199.5 g water was added to the reactor and purged with nitrogen with the aid of a sonicator. After the degassing and sonication step, a mixture of ethylenegycol dimethacrylate EGDMA (5.0 g), initiator (AIBN: 0.2 g, 0.095 wt %, 2 wt % of oil phase) and butyl acetate (5.0 g) as a non-solvent was introduced to the reaction flask and degassing was continued with slow stirring at 200 rpm for another 30 minutes. Reaction was started by ramping the temperature (20° C.) from room temperature to 70° C. in 1 hour. After a reaction time of 20 hours the microspheres were filtered off and washed with water and acetone.

### Results:

Three non-solvents were employed (dodecyl acetate DA, butyl acetate BA and ethyl acetate EA). While DA only resulted in agglomerated products, BA and EA result in hollow particles. BA was observed to be the most suitable solvent leading to hollow spheres with narrow particle size distribution. Variation of EGDMA/BA ratio allowed control over the wall thickness (FIG. 2).

The loading capacity of these spheres were demonstrated using Y(NO₃)₃. The particles were suspended in a concentrated solution of Y(NO₃)₃ in THF. After 5 days the particles were removed and washed with water to removed absorbed metal salt on the surface. The metal content was determined using TGA (thermogravimetric analysis). A metal content of 10% was calculated.

### Example 3

### Highly Crosslinked Microspheres: Formation of Poly(EGDMA) Hollow Spheres with BuAc as Solvent

Suspension polymerization: A diagrammatic representation of the reaction is shown in FIG. 5. In a typical suspension polymerization, a 250 ml glass reactor with 5 necks was used. The reactor was modified to include four 10 mm radial baffles. The aqueous phase was prepared by dissolving 1.05 g of the stabilizer, poly(N-vinyl pyrrolidone) (PVP) in 199.5 g of distilled water. The aqueous phase was then transferred to the reactor and was purged with nitrogen with the aid of an ultrasonic tip for 30 min. The dispersed phase comprised BuAc (8.4 g), EGDMA (2.1 g) and AIBN (0.0525 g). After sonification of the aqueous phase, the dispersed phase was introduced to the reactor. Nitrogen purging was continued for another 30 min. The reaction was then allowed to proceed at 70° C. at a stirring speed of 700 rpm for a period of 20 hours. The percentage of EGDMA in the mixture with BuAc was varied in this experiment. The yield was calculated based on the weight percentage of the product divided by the total weight of monomer and crosslinker used. The effect of this variation on spheres' diameters and wall thickness was examined.

| | | | | Wall | Diameter |
|---|---|---|---|---|---|
| EGDMA | BuAc | Yield | Diameter of | thickness | of hole |
| (wt %) | (wt %) | (%) | sphere (μm) | (μm) | (μm) |
| 50 | 50 | 96 | 120 | 30 | 60 |
| 60 | 40 | 90 | 143 | 40 | 63 |
| 75 | 25 | 93 | 198 | 59 | 80 |
| 85 | 15 | 81 | 157 | 48 | 61 |
| 95 | 5 | 92 | 81 | 28 | 25 |

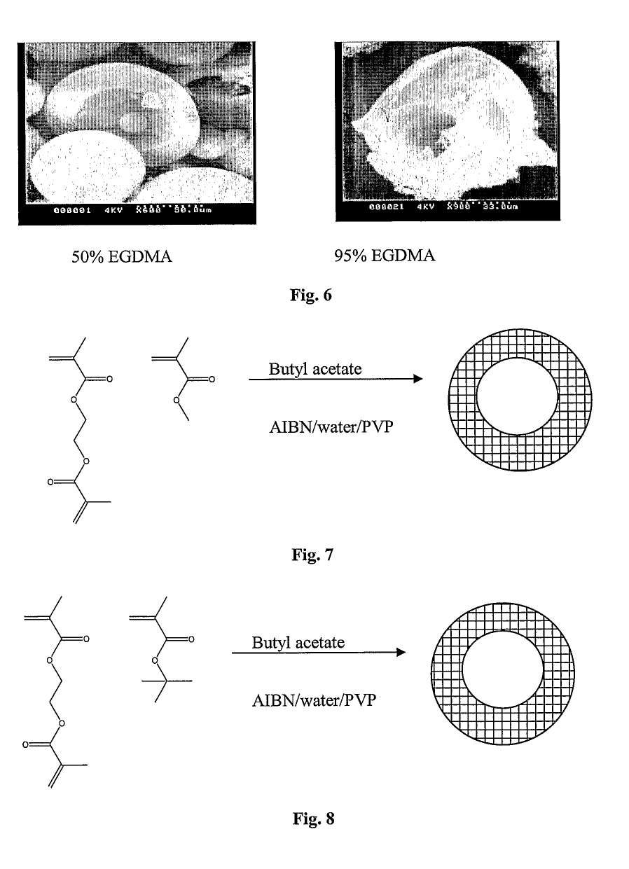

Electron micrographs of the spheres made using 50% and 95% EGDMA are shown in FIG. 6.

It appears that there is no simple correlation between the proportions of EGDMA and BuAc and the wall thickness. The mixture seems to affect the amount of particles and consequently the diameter of the particles. This may be because of the altered viscosity of the oil droplet. Lowering the proportion of EGDMA relative to BuAc resulted in collapsed structures.

### Example 4

### Microspheres with Reduced Crosslinking Density: Formation of poly(MMA co-EGDMA) Hollow Spheres

A diagram of the reaction of Example 4 is shown in FIG. 7. The suspension polymerization was carried out in a similar procedure as described in Example 3. In Example 4, a mixture of methylmethacrylate (MMA) and ethylene glycol dimethacrylate (EGDMA) in BuAc was used to create hollow spheres. Four mixtures of MMA/EGDMA/BuAc with a total weight of 10.5 g were prepared. The reaction was is carried out at 70° C. and 700 rpm for 20 h.

| | | | | | | Diameter |
|---|---|---|---|---|---|---|
| EGDMA | MMA | BuAc | Yield | Diameter | Wall | of hole |
| (w-%) | (w-%) | (w-%) | (%) | (μm) | thickness | (μm) |
| 10 | 40 | 50 | 52 | 58 | No hollow | |
| | | | | | spheres | |
| 20 | 30 | 50 | 70 | 82 | No hollow | |
| | | | | | spheres | |
| 30 | 20 | 50 | 80 | 66 | 20 | 26 |
| 40 | 10 | 50 | 100 | 62 | 19 | 24 |

It appears that a certain amount of crosslinker is necessary to stabilize the hollow spheres. However the minimum amount of crosslinker needed to produce hollow spheres appears also to be dependent on the nature of the crosslinker. A later example demonstrates production of hollow spheres with a crosslinker concentration as low as 5%.

### Example 5

### Hydrophilic Microspheres: Formation of Poly (tBuMA-co-EGDMA) Hollow Spheres

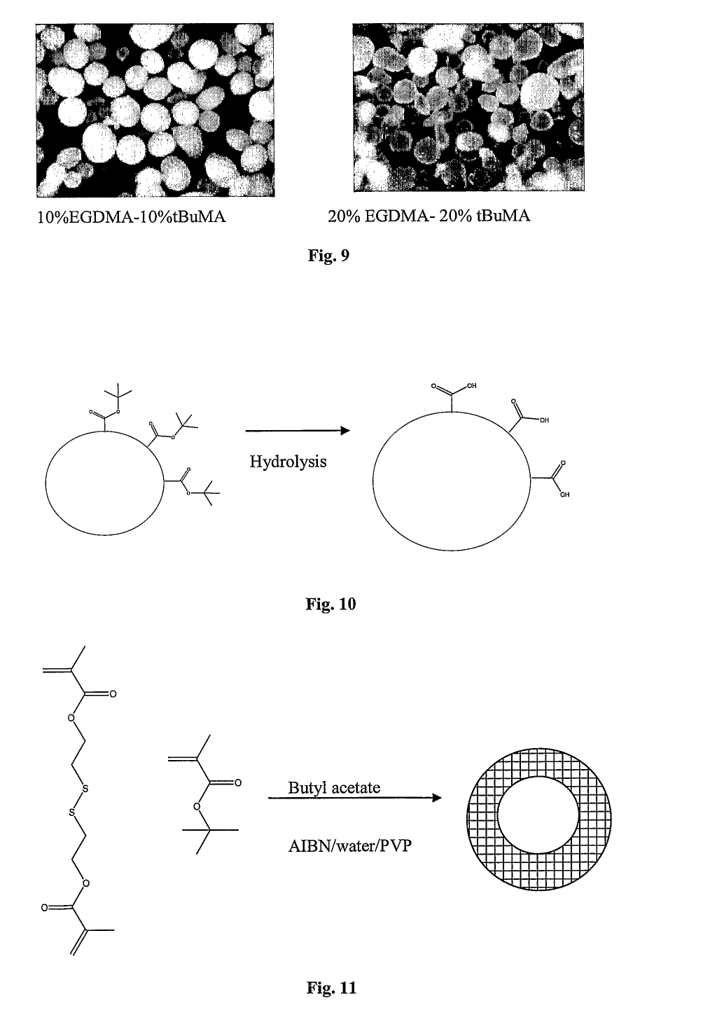

A diagram of the reaction of Example 5 is shown in FIG. 8. Suspension polymerization was carried out in a similar procedure as described in Example 3. In this example, a mixture of tert-butyl methacrylate (tBuMA) and ethylene glycol dimethacrylate (EGDMA) in BuAc was used to create hollow spheres. Various mixtures of tBuMA/EGDMA/BuAc with a total weight of 10.5 g were prepared. The amount of BuAc was kept unchanged at 50% while the percentages of the monomer and crosslinker were varied. The reaction was carried out at 70° C. and 750 rpm for 20 h.

| | | | | | Wall | Diameter |
|---|---|---|---|---|---|---|
| EGDMA | tBuMA | BuAc | Yield | Diameter | thickness | of hole |
| (w-%) | (w-%) | (w-%) | (%) | (μm) | (μm) | (μm) |
| 10 | 10 | 80 | 90 | 60 | 13 | 44 |
| 10 | 40 | 50 | 76 | 67 | 17 | 33 |
| 20 | 20 | 60 | 90 | 50 | 15 | 20 |
| 20 | 30 | 50 | 88 | 90 | 19 | 52 |
| 30 | 20 | 50 | 90 | 56 | 14 | 28 |

FIG. 9 shows micrographs of spheres produced using 10% EGDMA-10% tBuMA and 20% EGDMA-20% tBuMA

### Example 6

### Conversion of Hydrophobic into Hydrophilic Hollow Microspheres

A diagram of the reaction of Example 6 is shown in FIG. 10.

i) Hydrolysis a: A mixture of 8 mL of dioxane and 2 mL of 10% HCl was prepared. To this mixture, 100 mg of particles was added. The resulting heterogeneous mixture was heated at 80° C. in an oil bath for 24 h with stirring. After the reaction, the particles were filtered and washed with water and acetone. Particles were then air-dried and weighed. The percentage weight loss was subsequently calculated to determine the extent of the hydrolysis.

E=EGDMA, T=^{t}BuMA, D=bis(2-methacryloyloxyethyl)disulfide, B=butyl acetate

| Composition of monomer | | degree of |
|---|---|---|
| mixture of microsphere | % weight loss | hydrolysis (%) |
| 10% E - 40% T - 50% B | 14.8 | 34.5 |
| 20% E - 20% T - 60% B | 9.6 | 19.9 |
| 20% E - 30% T - 50% B | 8.9 | 19.2 |
| 30% E - 20% T - 50% B | 14.9 | 29.9 |
| 5% E - 50% T - 45% B | 4.0 | 9.8 |
| 5% D - 50% T - 45% B | 7.3 | 17.8 |

ii) Hydrolysis b: A mixture of 2 mL trifluoroacetic acid and 8 mL of chloroform was prepared. 0.1 g of the microspheres was suspended in this mixture. The hydrolysis was carried out at room temperature for 2 days under stirring. Upon collection, the product was washed thoroughly with acetone and oven dried.

| | | degree of |
|---|---|---|
| Polymer | % weight loss | hydrolysis (%) |
| 5% D - 50% T - 45% B | 36.0 | 90% |

### Example 7

### Formation of Peptide-Induced Degradable Hollow Spheres

A diagram of the reaction of Example 7 is shown in FIG. 11.

i) Synthesis of bis(2-methacryloyloxyethyl)disulfide: Bis(2-hydroxyethyl)disulfide (BHEDS, 7.7 g, 50 mmol) and triethylamine (50 mL, 40 mmol) were dissolved in 150 mL of anhydrous THF. The solution was then immersed in an ice bath and methacryloyl chloride (21 g, 200 mmol) was added dropwise to the stirred THF solution. The reaction was carried out at 20° C. for 24 h. Upon the completion of the reaction, the heterogeneous mixture was filtered to remove the solid triethylamine hydrochloride byproduct. THF was is removed by the rotary evaporator and the crude product was dissolved in chloroform. This solution was then washed three times with 0.1M Na₂CO₃ solution followed by three times with water. The resulting product was dried using anhydrous MgSO₄ and chloroform was removed by the rotary evaporator followed by a column separation and a Schlenk line. The conversion of this reaction was calculated based on ¹H-NMR analysis.
ii) Suspension polymerization: In this example, a mixture of bis(2-methacryloyloxyethyl)disulfide (disulfide dimethacrylate (DSDMA)) and ethylene glycol dimethacrylate (EGDMA) in BuAc was used to create hollow spheres. Various mixtures of DSDMA/EGDMA/BuAc with the total weight of 10.5 g were prepared. The suspension polymerization was carried out in a similar procedure to that described in Example 3. The reaction was carried out at 70° C. and 750 rpm for 20 h.

| | | | | | | Wall |
|---|---|---|---|---|---|---|
| DSDMA | MMA | ^{t}BuMA | BuAc | Yield | Diameter | thickness |
| (wt %) | (wt %) | (wt %) | (wt %) | (%) | (μm) | (μm) |
| 5 | 50 | — | 45 | 10 | 45 | 11 |
| 5 | — | 50 | 45 | 55 | 60 | 15 |

iii) Degradation: Glutathione (0.05 g) was dissolved in 5.0 mL of deionized water. Poly(tBuMA-DSDMA) microspheres (0.4 g) from ii above were then suspended in this solution and purged for 30 min with nitrogen. The reduction reaction between glutathione and disulfide bonds in microspheres was allowed to proceed at 37° C. or 60° C. in nitrogen environment for 7 days. After reaction, samples were filtered and washed with water. The powder was collected and re-suspended in acetone and then washed with acetone 3 times. Particles were air dried and weighed to determine the percentage of weight loss.

| Reaction Temperature | % weight loss |
|---|---|
| 37° C. | 11% |
| 60° C. | 14% |

### Example 8

### Formation of pH-Sensitive Hollow Spheres

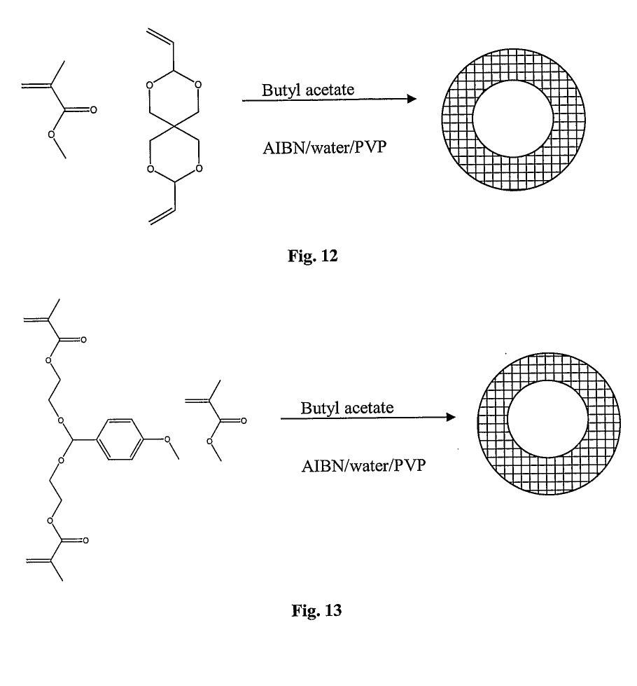

Approach a): the reaction of approach a) is shown in FIG. 12.
i) Suspension polymerization. In this example, a mixture of the acid degradable is crosslinker (AC: 3,9-Divinyl-2,4,8,10-tetraoxaspiro[5.5]undecane) and tert-butyl methacrylate (tBuMA) in BuAc was used to create hollow spheres. A mixture contains of the crosslinker (0.32 g), tBuMA (2.88 g), BuAc (6.8 g) and AIBN (0.05 g) was prepared. The reaction was carried out at 70° C. and 750 rpm for 20 h. A yield of 91% was obtained. No well defined microspheres were obtained.
ii) Degradation: 200 mg of the product from (i) above was suspended in 5 mL of the buffered solution at pH 5.0 to study the effect of the pH condition on the degradation of microspheres. The hydrolysis reaction was allowed to proceed at 37° C. and 60° C. for 7 days. After reaction, the particles were collected from the filtration process and washed with water and acetone extensively. Percentage of weight loss was determined gravimetrically.

| Hydrolysis Temperature | % weight loss |
|---|---|
| 37° C. | 24% |
| 60° C. | 24% |

### Approach b)

The reaction of approach b) is shown in FIG. 13.

i) Synthesis of crosslinker: p-Methoxybenzaldehyde (0.0485 mol) and HEMA (hydroxyethylmethacrylate) (0.34 mol) were reacted in an ice-bath using p-toluene sulfonic acid (0.0084 mol) as catalyst. After 24 h, the reaction was quenched by adding triethylamine (0.0072 mol). The resulting solution was then extracted with 0.1M aqueous potassium carbonate solution. Extra extraction with diethyl ether was desirable to remove residual HEMA. After extraction, diethyl ether was removed from the organic mixture by means of a rotary evaporator, leaving behind the product. The product was then passed through a basic alumina column to remove the inhibitor.
ii) Suspension polymerization. In this example, a mixture of the acid degradable crosslinker (AC) and tert-butyl methacrylate (tBuMA) in BuAc was used to create hollow spheres. A mixture containing of the crosslinker from i) above (1.0 g), tBuMA (4.0 g), BuAc (5 g) and AIBN (0.05 g) was prepared. The reaction was carried out at 70° C. and 750 rpm for 40 h. A yield of 23% was obtained.
iii) Degradation: 200 mg of microspheres from ii) above was suspended in 5 mL of a citrate-phosphate buffered solution at pH 5.0 to study the effect of pH on the degradation of microspheres. The hydrolysis reaction was allowed to proceed at 37° C. and 60° C. for 4 days. After reaction, the particles were collected from the filtration process and washed with water and acetone extensively. Percentage weight loss was determined gravimetrically.

| Hydrolysis Temperature | % weight loss |
|---|---|
| 37° C. | 14% |
| 60° C. | 28% |

1. A process for making hollow microparticles comprising:
a) providing a dispersion having a continuous aqueous phase and a discontinuous organic phase, wherein the continuous aqueous phase comprises a stabiliser and the discontinuous organic phase comprises a monomer having two or more polymerisable groups per molecule and an organic liquid; and
b) polymerising the monomer in the dispersion to form hollow polymeric microparticles;
wherein, prior to step b) the discontinuous organic phase does not contain a polymer.

2. The process of claim 1 wherein the monomer is selected so that the polymer of the microparticles is capable of reacting with a chemical to which the microparticles are exposed in order to release a substance encapsulated in the hollow polymeric microparticles.

3. The process of claim 2 wherein the monomer comprises a cleavable linkage between two of the polymerisable groups, such that the polymer of the microparticles is capable of degrading in order to release the substance.

4. The process of claim 3 wherein the cleavable linkage is a hydrolysable linkage and the polymer is capable of hydrolysing in order to release the substance.

5. The process of any one of claims 1 to 3 wherein the cleavable linkage is selected from the group consisting of a disulfide, an ester, an anhydride, an orthoester and an acetal.

6. The process of any one of claims 1 to 5 wherein the discontinuous organic phase additionally comprises a second monomer which is capable of copolymerising in step b) with the monomer having two or more polymerisable groups per molecule.

7. The process of claim 6 wherein the monomer having two or more polymerisable groups per molecule comprises a degradable non-hydrolysable linking group and the second monomer comprises a hydrolysable group, whereby a shell of the hollow microparticles comprises a copolymer comprising degradable non-hydrolysable crosslinks and also comprising hydrolysable groups.

8. The process of claim 7 additionally comprising at least partially hydrolysing the hydrolysable groups of the copolymer so as to render the surface of the microparticles hydrophilic.

9. The process of any one of claims 1 to 8 wherein the monomer having two or more polymerisable groups per molecule or the second monomer, if present, or both, comprises a hydrolysable group, such that hydrolysis of the hydrolysable group in the polymer of the microparticles alters the polarity of said polymer without causing backbone chain breaking of the polymer.

10. The process of any one of claims 1 to 9 wherein the stabiliser is polymeric.

11. The process of any one of claims 1 to 10 wherein the stabiliser is a thickener.

12. The process of any one of claims 1 to 11 wherein the organic liquid is a non-solvent for the polymer.

13. The process of any one of claims 1 to 12 wherein the organic liquid is a solvent for the monomer having two or more polymerisable groups per molecule and for the second monomer, if present.

14. The process of any one of claims 1 to 13 wherein the discontinuous organic phase comprises a thermal initiator and step b) comprises heating the dispersion so as to polymerise the monomer(s).

15. The process of any one of claims 1 to 14 wherein the discontinuous organic phase comprises a photoinitiator and step b) comprises irradiating the dispersion with radiation having a wavelength and intensity sufficient to polymerise the monomer(s).

16. The process of any one of claims 1 to 15 additionally comprising:
c) loading a substance into the interior region of the hollow microparticles to form loaded microparticles.

17. The process of claim 16 wherein the substance is a drug.

18. The process of claim 16 or 17 wherein the substance is usable in the treatment of cancer.

19. The process of any one of claims 16 to 18 wherein neutron bombardment of the substance generates a n-emitter.

20. Hollow microparticles made by the process of any one of claims 1 to 19.

21. A method of treating a condition in a patient comprising administering to said patient a therapeutically effective quantity of microparticles according to claim 20, wherein a substance indicated for treatment of said condition is located in the interior region of the microparticles.

22. A method of treating cancer in a patient comprising:
exposing a therapeutic quantity of hollow polymeric microparticles according to claim 20 to neutrons from a neutron source, wherein a substance in the interior region of the hollow polymeric microparticles is such that the exposure generates a β-emitter; and
administering the therapeutic quantity of hollow microparticles to the patient.

23. Use of hollow polymeric microparticles according to claim 20 for the manufacture of a medicament for the treatment of cancer.

24. A method for releasing a substance from a core of a polymeric microparticle according to claim 20, said core containing the substance, said method comprising exposing said polymeric microparticle to a reagent which causes cleavage of crosslinks of a polymeric shell of the microparticle so as to cause said microparticle to release said substance.

25. The method of claim 24 wherein said crosslinks are hydrolysable crosslinks and said reagent is a hydrolytic reagent.

26. The method of claim 25 wherein said crosslinks are disulfide crosslinks and said reagent comprises a thiol.
